(19) United States
(12) Patent Application Publication
(10) Pub. No.: US 2009/0023606 A1
(43) Pub. Date: Jan. 22, 2009

(54) Beaded and Cross-Linked Poly(Aminoalkylene)Matrix and Uses Thereof

(75) Inventors: Patrik Gavelin, Rydeback (SE); Nicola Pehr-Rehnberg, Perstorp (SE); Ib Johannsen, Vaerlose (DK)

Correspondence Address: EDWARDS ANGELL PALMER & DODGE LLP, P.O. BOX 55874, BOSTON, MA, 02205, US

(73) Assignee: VERSAMATRIX A/S, Valby (DK)

(21) Appl. No.: 11/659,863

(22) PCT Filed: Jun. 10, 2005
(86) PCT No.: PCT/DK05/00384

§ 371 Date: Mar. 31, 2008

Related U.S. Application Data
(60) Provisional application No. 60/600,800, filed on Aug. 12, 2004.

(30) Foreign Application Priority Data
Aug. 12, 2004 (DK) PA 2004 01220

(51) Int. Cl. C08F 226/02; C40B 50/14; C07K 1/00; C07K 1/04; C07H 21/02; C07H 21/04

(52) U.S. Cl. 506/30; 525/326.1; 526/310; 530/334; 530/350; 536/23.1

(57) ABSTRACT

The present invention relates to the synthesis of a beaded and cross-linked, high loading capacity polymer for solid phase synthesis, purification of reaction mixtures, chromatographic separation procedures, and the like. The invention can thus be used for the isolation of molecular entities having an affinity for the polymer beads or a chemical entity attached thereto. The beaded polymer matrix can be formed by cross-linking an optionally substituted poly(aminoalkylene), under inverse suspension or inverse emulsion polymerisation conditions, with a cross-linking unit of functionality ≧2.

### FIELD OF THE INVENTION

The present invention relates to the synthesis of a beaded and cross-linked, high loading capacity polymer for solid phase synthesis, purification of reaction mixtures, chromatographic separation procedures, and the like. The invention can thus be used for the isolation of molecular entities having an affinity for the polymer beads or a chemical entity attached thereto.

### BACKGROUND OF THE INVENTION

The interest and importance of solid phase chemistry technology has rapidly evolved during several decades. The early investigations of its use focused on the synthesis of oligomers of amino acids or nucleotides, or on unnatural oligomers of other chemical building blocks like peptoids [Geysen et al., J. Bioorg. Med. Chem. Lett., 3, 397 (1993); Egholm et al., J. Am. Chem. Soc., 114, 1895 (1992); Simon et al., Proc. Natl. Acad. Sci. USA, 89, 9367 (1992)]. In recent years, library synthesis of non-oligomeric small molecules has become an area of intense research activity [Wang et al., J. Med. Chem., 38, 2995 (1995)].

In all approaches to produce chemical products, whether solid-phase or solution-phase, is the need of high loading capacity, rapid purification, and isolation. The solid phase technology offers advantages like ease of separating the products from the reaction medium and the handling of the beads using volumetric techniques. The limitation of solid-phase technology includes the reaction scale restriction, frequent low bead capacity, the need of product validation, and the decrease of reactivity inherent to solid phase synthesis. Solution phase synthetic technology has the advantage of non-limiting scale. The major limitation of solution-phase synthesis is the isolation or purification of the reaction products from the reaction mixture particularly when working with complex products.

The use of polymeric beaded synthesis supports and polymeric beaded scavenging functions of the present invention can overcome this limitation to a great extent. The rational behind this concept is that the present resins containing high functional group density and, in its basic concept, amino functional groups that are very versatile as they are easily converted into a plethora of other functional groups.

U.S. Pat. No. 4,605,701 discloses cross-linked homopolymers of monoallylamine.

JP 61-051007 published on 13 Mar. 1986 discloses cross-linked polyvinylamines.

WO 03/08503 discloses swellable, easily cross-linked, essentially linear polymers and the use thereof.

WO 00/55258 discloses mixed bed ion-exchange absorbent polymer compositions.

### SUMMARY OF THE INVENTION

The beaded polymer matrices according to the present invention can be utilized as insoluble supports in chemical or biochemical synthesis, peptide synthesis, oligonucleotide synthesis, oligosaccharide synthesis, catalysis applications, affinity chromatography, pharmaceutical applications, for enzyme immobilization, and for scavenging chemical moieties, such as e.g. carbonyl moieties or acid chlorides.

The chemical synthesis of compounds with the use of the concept of combinatorial libraries has an important influence on the process of developing potential candidates for new therapeutic and diagnostic agents. Combinatorial chemistry is a technique in which a large number of structurally different compounds are produced under comparable reaction conditions in a cost favourable and time efficient manner. The compounds can subsequently be introduced into biological testing by high performance screening assays.

The use of the polymer matrices according to the invention as reagent supports, e.g. in organic or bioorganic reactions, facilitates the separation of products and reagents and has other advantages such as e.g. scavenging undesirable by-products.

The is also provided a functional surface comprising the polymer matrix comprising a high functional group density, preferably primary amines or derivatives thereof, wherein said functional groups are attachment sites for solid phase synthesis reagents, linkers, spacers, intermediates or end products.

In one aspect there is provided a beaded polymer matrix, formed by cross-linking of optionally substituted poly(aminoalkylene), under inverse suspension or inverse emulsion polymerisation conditions, of Formula I

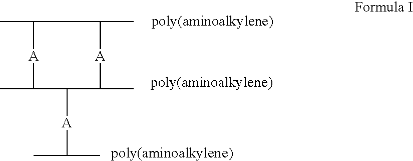

wherein A is a cross-linking unit of functionality ≧2,
with the proviso that at least 1% of all nitrogens are substituted when the poly(aminoalkylene) is poly(allylamine), and
with the further proviso that when the poly(aminoalkylene) is poly(vinylamine) then A is not
(a) a polymethylene of the formula (CH₂)ᵣ, wherein r is an integer from 2 to 10, or
(b) an optionally substituted xylylene, or
(c) a diimine linked by a polymethylene of the formula (CH₂)ₛ wherein s is an integer from 2 to 5, or
(d) a diimine linked by an optionally substituted xylylene, or

### (e) CH₂CHOHCH₂, or CH₂CHCH₂OH.

In another aspect there is provided a beaded and cross-linked poly(aminoalkylene) matrix obtained by radical polymerization of a molecule of Formula IV having a radical reactive group R⁴R″C═CR″C═Y

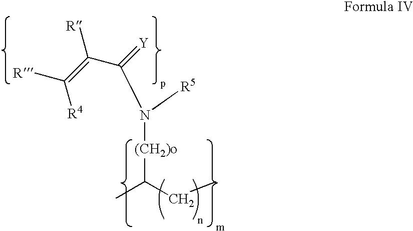

wherein
n is a number from 0 to 10;
m is a number from 3 to 15000;
o is number 0 or 1;
p is a number >0 and <m;
Y is a heteroatom; and
R″, R′″, R⁴, and R⁵ are independently selected from the group consisting of hydrogen, optionally substituted saturated or unsaturated alkyl groups, and optionally substituted aryl groups.

Uses of—and methods for generating—the above-mentioned beaded and cross-linked matrices are also provided in accordance with the present invention.

Methods for generating the above-mentioned beaded and cross-linked matrices include radical polymerization methods.

When the polymer matrices are made by radical polymerization methods, there is further provided in accordance with the present invention a polymer matrix comprising a plurality of substituted amino groups, wherein the polymer matrix is obtained by a radical polymerization method as disclosed herein in combination with the further step of converting—after the polymerisation and beading steps—at least some of the amino groups to functional groups NR⁶R⁷, of Formula V:

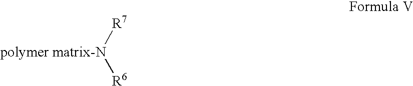

wherein R⁶ and R⁷ independently are selected from the group consisting of hydrogen and an organic group formed by reaction of the amino groups of the polymer matrix according to the invention with an alkylating or acylating agent.

### DEFINITIONS

Inverse emulsion polymerisation—see “Principles of Polymerisation”, 3 ed, George Odian, John Wiley & Sons, Inc., NY, 1991, ISBN 0-471-61020-8.

Inverse suspension polymerisation—see “Principles of Polymerisation”, 3 ed, George Odian, John Wiley & Sons, Inc., NY, 1991, ISBN 0-471-61020-8.

The terms “saturated or unsaturated alkyl group” and “saturated or unsaturated aliphatic group” are intended to mean an aliphatic group having one or more unsaturated carbon atom pairs. Examples hereof are methyl, ethyl, propyl, i-propyl, allyl, butyl, i-butyl, etc.

The term “alkyl group” is intended to mean a saturated aliphatic group, e.g. methyl, ethyl, propyl, i-propyl, butyl, i-butyl, etc.

The term “aryl group” is intended to mean an aromatic group having one or more rings, e.g. phenyl, naphthyl, etc.

The term “arylalkyl group” is intended to mean an alkyl group carrying an aryl group, e.g. benzyl, p-methoxybenzyl, etc.

The term “acyl group” is intended to mean a group of the formula R—C(═O)—, wherein R is selected from the group consisting of optionally substituted saturated or unsaturated alkyl groups and optionally substituted aryl groups, etc. Examples of acyl groups are formyl, acetyl, propanoyl, acryloyl, butanoyl, i-butanoyl, ethoxyacetyl, benzoyl, p-methoxybenzoyl, naphthoyl, nicotinoyl, etc.

Alkyl groups preferably have from 1 to 10 carbon atoms, saturated or unsaturated aralkyl groups typically have from 1 to 10 carbon atoms, and saturated or unsaturated acyl groups typically have 1 to 10 carbon atoms, said groups optionally having from 1 to 4 heteroatoms such as nitrogen, oxygen, or sulphur.

The term “optionally substituted” is intended to mean that the group in question may carry one or more substituents.

### DETAILED DESCRIPTION OF THE INVENTION

In one embodiment, the optionally substituted poly(aminoalkylene) can be illustrated by Formula II

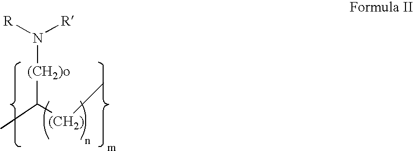

wherein
R and R′ are independently selected from the group consisting of hydrogen, optionally substituted alkyl groups, optionally substituted aryl groups, and optionally substituted acyl groups;
n is an integer from 0 to 10; for example from 0 to 4, such as from 0 to 2, for example 0 or 1;
m is an integer from 3 to 15000 such as an integer from 5 to 15000, for example an integer from 50 to 10000, such as an integer from 100 to 10000, for example an integer from 100 to 8000, such as an integer from 100 to 7000, for example an integer from 100 to 6000, such as an integer from 100 to 5000, for example an integer from 100 to 4500, such as an integer from 100 to 4000, for example an integer from 100 to 3500, such as an integer from 100 to 3000, for example an integer from 100 to 2000, such as an integer from 100 to 1500, for example an integer from 100 to 1000, such as an integer from 100 to 500, for example an integer from 500 to 10000, such as an integer from 1000 to 10000, for example an integer from 1500 to 10000, such as an integer from 2000 to 10000, for example an integer from 2500 to 10000, such as an integer from 3000 to 10000, for example an integer from 3500 to 10000, such as an integer from 4000 to 10000, for example an integer from 4500 to 10000, such as an integer from 5000 to 10000, for example an integer from 5500 to 10000, such as an integer from 6000 to 10000, for example an integer from 6500 to 10000, such as an integer from 7000 to 10000, for example an integer from 7500 to 10000, such as an integer from 8000 to 10000, for example an integer from 9000 to 10000, such as an integer from 9500 to 10000, for example an integer from 500 to 1000, such as an integer from 1000 to 1500, for example an integer from 1500 to 2000, such as an integer from 2000 to 2500, for example an integer from 2500 to 3000, such as an integer from 3000 to 3500, for example an integer from 3500 to 4000, such as an integer from 4000 to 4500, for example an integer from 4500 to 5000, such as an integer from 5000 to 5500, for example an integer from 5500 to 6000, such as an integer from 6000 to 6500, for example an integer from 6500 to 7000, such as an integer from 7000 to 7500, for example an integer from 7500 to 8000, such as an integer from 8000 to 8500, for example an integer from 8500 to 9000, such as an integer from 9000 to 9500, for example an integer from 9500 to 10000, such as an integer from 1000 to 2000, for example an integer from 2000 to 3000, such as an integer from 3000 to 4000, for example an integer from 4000 to 5000, such as an integer from 5000 to 6000, for example an integer from 6000 to 7000, such as an integer from 7000 to 8000, for example an integer from 8000 to 9000, such as an integer from 1000 to 5000, for example an integer from 2500 to 7500, such as an integer from 200 to 250, for example an integer from 950 to 1150, such as an integer from 7500 to 8500; and
wherein o is 0 or 1, e.g. 0 or 1.

The integer m represent the average degree of polymerisation and is the value corresponding to a poly(aminoalkylene) species having the average molecular weight for the batch of material.

In one embodiment, R and R′ are independently selected from the group consisting of hydrogen, alkyl groups and acyl groups.

In another embodiment, R and R′ are independently selected from the group consisting of saturated or unsaturated aliphatic groups, saturated or unsaturated arylalkyl groups having from 1 to 15 carbon atoms, and optionally having from 1 to 4 heteroatoms, such as nitrogen, oxygen, or sulphur, and saturated or unsaturated acyl groups having from 1 to 15 carbon atoms, optionally having 1-4 heteroatoms such as nitrogen, oxygen, or sulphur.

R and R′ are preferably independently selected from the group consisting of methyl, ethyl, propyl, i-propyl, allyl, butyl, i-butyl, ethoxyethyl, benzyl, p-methoxybenzyl, naphthyl, formyl, acetyl, propanoyl, acryloyl, butanoyl, i-butanoyl, ethoxyacetyl, benzoyl, p-methoxybenzoyl, naphthoyl, and nicotinoyl.

In preferred embodiments, the poly(aminoalkylene) is optionally substituted poly(aminomethylene), optionally substituted polyvinylamine, or substituted poly(allylamine), with the provisos listed herein above.

Preferably, when the poly(aminoalkylene) is poly(allylamine), at least 2% of all nitrogens are substituted, such as at least 5%, for example 8%, such as 10%, for example 15%, such as 20%, for example 30%, such as 40%, for example 50%, such as 60%, for example 70%, such as 80%, for example 90%, such as 95%, for example essentially all nitrogens are substituted. The degree of nitrogen substitution can also be e.g. from 1% to 25%, from 25% to 50%, from 50% to 75%, and from 75% to 100%.

When the poly(aminoalkylene) is not poly(allylamine), the nitrogens can be optionally substituted.

In one embodiment, when the poly(aminoalkylene) is poly(aminomethylene), poly(vinylamine), or poly(allylamine), part of the nitrogens of the beaded cross-linked polymer are substituted, such as from 1% to 20%, for example from 20% to 40%, such as from 40% to 60%, for example from 60% to 80%, such as from 80% to 100%, of all nitrogens are substituted.

When the beaded polymer matrix of Formula I is obtained by cross-linking an optionally substituted poly(aminoalkylene) under inverse suspension or inverse emulsion polymerisation conditions, the cross-linking unit A has a functionality of 2 or more and is preferably obtained by reacting a poly(aminoalkylene) with a cross-linking molecule of Formula III

AX_{q} Formula III,

wherein
A is a saturated or unsaturated aliphatic or aromatic, or composed of both saturated and/or unsaturated aliphatic and aromatic fragments, and optionally containing heteroatoms such as silicon, nitrogen, phosphorous, oxygen, or sulphur;
X is a reactive group;
q, is the number of reactive groups, such as e.g. from 2 to 10, preferably 2, 3, 4, 5, or 6;
with the proviso that when poly(aminoalkylene) is poly(vinylamine), AX_{q} is not
(a) a dibrominated or diiodated polymethylene expressed by general Formula (2)

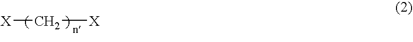

where X denotes Br or I, and n′ denotes an integer of 2 to 10, or
(b) a p-dihalogenated xylylene expressed by general Formula (3)

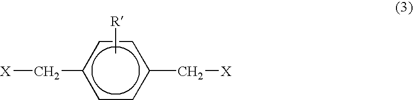

where X denotes Cl, Br, or I; and R′ denotes H, a methyl group, an ethyl group, or a halogen atom, or
(c) a nuclear-substituted derivative thereof capable of binding an optionally alkyl-substituted primary amino group, or
(d) a polymethylene dialdehyde expressed by general Formula (4)

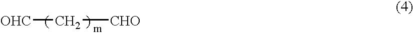

where m denotes an integer of 2-5, or
(e) a dialdehyde having an intramolecular benzene nucleus expressed by general Formula (5)

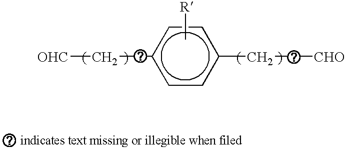

where I denotes 0 or an integer of 1-20, and R′ denotes H, a methyl group, an ethyl group, or a halogen atom, or
(f) epichlorohydrin.

A is preferably an aliphatic group or an alkylaryl group having from 2 to 200 carbon atoms, and optionally having from 1 to 100 hetero atoms such as nitrogen, oxygen, or sulphur; preferably an aliphatic or alkylaryl group having 10 to 100 carbon atoms and optionally having 2 to 50 hetero atoms, such as nitrogen, oxygen, or sulphur.

A is preferably selected from the group consisting of 1,2-ethylene, 1,3-propylene, 1,4-butylene, 1,4-butenylene, 1,5-pentylene, 1,6-hexylene, o-xylylene, p-xylylene, oxydiethyl, tri(ethylene oxide)diyl, tetra(ethylene oxide)diyl, penta(ethylene oxide)diyl, hexa(ethylene oxide)diyl, hepta(ethylene oxide)diyl, octa(ethylene oxide), nona(ethylene oxide)diyl, deca(ethylene oxide)diyl, and a polydisperse poly(ethylene oxide)diyl, such as (ethylene oxide)₁₀diyl, polydisperse (ethylene oxide)₁₅diyl, polydisperse (ethylene oxide)₂₀diyl, polydisperse (ethylene oxide)₂₅diyl, polydisperse (ethylene oxide)₃₀diyl, polydisperse (ethylene oxide)₄₀diyl, and polydisperse (ethylene oxide)₄₅diyl, or wherein A comprises one or more members of the above defined group, including any combination thereof.

The reactive group X of Formula III is preferably a reactive group selected from the group of reactive groups consisting of S_{N}2 leaving groups, Michael acceptors, isocyanates and carbonyl groups capable of undergoing reductive amination, with the proviso that the cross-linking step is followed by reduction of the imine to the amine.

When the reactive group X of Formula III is a S_{N}2 leaving group, preferred examples include chloride, bromide, iodide, methanesulfonate, trifluoromethanesulfonate, p-toluenesulfonate, or an epoxide.

When the reactive group X of Formula III is a Michael acceptor, preferred examples include acrylate, methacrylate, ethacrylate, or acrylamido.

Wherein the reactive group X of Formula III is a constituent of an aliphatic or aromatic molecule.

When the reactive group X of Formula III is a carbonyl group capable of undergoing reductive amination, with the proviso that the cross-linking step is followed by reduction of the imine to the amine, preferred examples include aldehydes and ketones.

Preferably, the reducing agent used for converting the imine to the amine comprises a borohydride such as sodium borohydride or sodium cyanoborohydride, or an aluminium hydride such as lithium aluminiumhydride or sodium bis(2-methoxyethoxy)aluminiumhydride.

Examples of AX_{q} include, but is not limited to, S_{N}2 leaving group compounds such as e.g. ethylene dibromide, propylene dibromide, butylene dibromide, xylylene dibromide, ethylene glycol ditosylate, diethylene glycol dichloride, triethyleneglycol dichloride, polyethylene glycol dichloride, epichlorohydrine, ethylene glycol diglycidyl ether, diethylene glycol diglycidyl ether, triethylene glycol diglycidyl ether, polydisperse polyethylene glycol diglycidyl ether such as (ethylene oxide)₁₀ diglycidyl ether, (ethylene oxide)₁₅ diglycidyl ether, (ethylene oxide)₂₀ diglycidyl ether, ethoxylated trimethylolpropane triglycidyl ether, ethoxylated dipentaerythritol hexaglycidyl ether, with the proviso that when AX_{q} is ethylene dibromide, propylene dibromide, butylene dibromide, xylylene dibromide then poly(aminoalkylene) is not an optionally substituted polyvinylamine; Michael acceptors such as e.g. ethylene glycol diacrylate, diethyleneglycol diacrylate, polyethylene glycol diacylate, polyethyleneglycol dimethacrylate, ethoxylated trimethylolpropane triacrylate, ethoxylated dipentaerythritol hexaacrylate, or Jeffamine diacrylate; isocyanates such as 1,6-hexane diisocyanate, isophorone diisocyanate, toluene diisocyanate, and 1,4-phenylene diisocyanate; and carbonyl compounds such as e.g. formaldehyde, glyoxal, succinaldehyde, glutaraldehyde, 1,4-diformylbenzene, 1,4-diacetylbenzene, polyethylene glycol di(formylmethyl)ether, with the proviso that the cross-linking step is followed by a reduction of the imine to the amine.

When the invention is directed to a beaded cross-linked poly(aminoalkylene) matrix obtained by radical polymerization of a molecule of Formula IV having a radical reactive group R⁴R″C═CR″C═Y

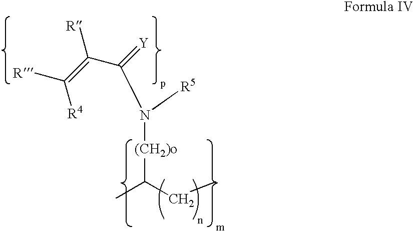

wherein
n is a number from 0 to 10;
m is a number from 3 to 50000 such as from 3 to 15000;
o is number 0 or 1;
p is a number >0 and <m;
Y is a heteroatom or a pair of hydrogen atoms; and
R″, R′″, R⁴, and R⁵ are independently selected from the group consisting of hydrogen, optionally substituted saturated or unsaturated alkyl, optionally substituted saturated or unsaturated acyl, and optionally substituted aryl groups.
n is preferably an integer from 0 to 10; for example from 0 to 4, such as from 0 to 2, for example 0 or 1; and independently thereof.
m is preferably an integer from 3 to 15000 representing an average molecular weight of polydisperse poly(aminoalkylene); such as an integer from 5 to 15000, for example an integer from 50 to 10000, such as an integer from 100 to 10000, for example an integer from 100 to 8000, such as an integer from 100 to 7000, for example an integer from 100 to 6000, such as an integer from 100 to 5000, for example an integer from 100 to 4500, such as an integer from 100 to 4000, for example an integer from 100 to 3500, such as an integer from 100 to 3000, for example an integer from 100 to 2000, such as an integer from 100 to 1500, for example an integer from 100 to 1000, such as an integer from 100 to 500, for example an integer from 500 to 10000, such as an integer from 1000 to 10000, for example an integer from 1500 to 10000, such as an integer from 2000 to 10000, for example an integer from 2500 to 10000, such as an integer from 3000 to 10000, for example an integer from 3500 to 10000, such as an integer from 4000 to 10000, for example an integer from 4500 to 10000, such as an integer from 5000 to 10000, for example an integer from 5500 to 10000, such as an integer from 6000 to 10000, for example an integer from 6500 to 10000, such as an integer from 7000 to 10000, for example an integer from 7500 to 10000, such as an integer from 8000 to 10000, for example an integer from 9000 to 10000, such as an integer from 9500 to 10000, for example an integer from 500 to 1000, such as an integer from 1000 to 1500, for example an integer from 1500 to 2000, such as an integer from 2000 to 2500, for example an integer from 2500 to 3000, such as an integer from 3000 to 3500, for example an integer from 3500 to 4000, such as an integer from 4000 to 4500, for example an integer from 4500 to 5000, such as an integer from 5000 to 5500, for example an integer from 5500 to 6000, such as an integer from 6000 to 6500, for example an integer from 6500 to 7000, such as an integer from 7000 to 7500, for example an integer from 7500 to 8000, such as an integer from 8000 to 8500, for example an integer from 8500 to 9000, such as an integer from 9000 to 9500, for example an integer from 9500 to 10000, such as an integer from 1000 to 2000, for example an integer from 2000 to 3000, such as an integer from 3000 to 4000, for example an integer from 4000 to 5000, such as an integer from 5000 to 6000, for example an integer from 6000 to 7000, such as an integer from 7000 to 8000, for example an integer from 8000 to 9000, such as an integer from 1000 to 5000, for example an integer from 2500 to 7500, such as an integer from 200 to 250, for example an integer from 950 to 1150, such as an integer from 7500 to 8500.

Independently of the before-mentioned, the number of reactive groups p per polymer chain is in the range of from 0.01 m<p<m, such as from 0.05 m<p<0.80 m, for example from 0.05 m<p<0.70 m, such as from 0.05 m<p<0.60 m, for example from 0.05 m<p<0.50 m, such as from 0.05 m<p<0.40 m, for example from 0.05 m<p<0.30 m, such as from 0.05 m<p<0.20 m, for example from 0.1 m<p<0.80 m, such as from 0.1 m<p<0.70 m, for example from 0.1 m<p<0.60 m, such as from 0.1 m<p<0.50 m, for example from 0.1 m<p<0.40 m, such as from 0.1 m<p<0.30 m, for example from 0.1 m<p<0.2 m, such as from 0.2 m<p<0.3 m, for example from 0.3 m<p<0.4 m, such as from 0.4 m<p<0.5 m, for example from 0.5 m<p<0.6 m, such as from 0.6 m<p<0.7 m, for example from 0.7 m<p<0.8 m, such as from 0.8 m<p<0.9 m, for example from 0.9 m<p<m.

The configuration Y can be any heteroatom, such as e.g. an oxygen atom, a sulphur atom, or a pair of hydrogen atoms, preferably an oxygen atom.

In Formula IV, R⁵ is preferably independently selected from the group consisting of hydrogen or formyl, and R″, R′″, and R⁴ are independently preferably selected from the group consisting of hydrogen, alkyl groups, aralkyl groups and aryl groups. For example, R″, R′″, R⁴, and R⁵ can be independently selected from the group consisting of hydrogen, saturated or unsaturated aliphatic groups having from 1 to 10 carbon atoms, saturated or unsaturated aralkyl groups having from 1 to 10 carbon atoms, and saturated or unsaturated aryl groups having 1 to 10 carbon atoms, said groups optionally having from 1 to 4 heteroatoms such as nitrogen, oxygen, or sulphur.

In one embodiment, R″, R′″, R⁴, and R⁵ can preferably be independently selected from the group consisting of hydrogen, methyl, ethyl, propyl, i-propyl, allyl, butyl, i-butyl, ethoxyethyl, benzyl, p-methoxybenzyl, naphthyl, formyl, acetyl, propanoyl, acryloyl, butanoyl, I-butanoyl, ethoxyacetyl, benzoyl, p-methoxybenzoyl, naphthoyl, or nicotinyl. Preferred examples of the reactive group R⁴R′″C═CR″—CY are acryloyl, methacryloyl, ethacryloyl, and allyl.

The poly(aminoalkylene) obtained by radical polymerization preferably comprises or consists of poly(aminomethylene), polyvinylamine, or poly(allylamine).

The present invention is also directed to methods for generating the above-mentioned beaded and cross-linked polymer matrices. The beaded cross-linked polymer matrices of the invention can be prepared e.g. by reacting a polyamine, or a derivative thereof, such as a substituted polyamine, with a multifunctional cross-linker under suspension polymerisation conditions. The polyamine may be poly(aminomethylene), poly(aminoethylene), or polyallylamine, or derivatives thereof. The multifunctional cross-linker can be e.g. a polyepoxide, a polyhalide, a polyisocyanate or a poly(Michael acceptor).

In one embodiment, the polyamine and the multifunctional cross-linker are mixed and a surface active agent is preferably added. Optionally, a solvent such as water, ethylene glycol, diethylene glycol, or dimethylformamide, or mixtures thereof, is added. This mixture is then added to a reactor containing a medium in which the reaction mixture is insoluble or essentially insoluble. The reactor is equipped with a stirring devise to efficiently form droplets of the reactive phase dispersed in the continuous phase. Optionally, the surface active agent may be added to the continuous phase instead of adding it to the reactive monomer phase. The temperature is adjusted so as to reach a reasonable reaction speed and reaction time. Optionally a catalyst such as a basic component exemplified by triethylamine or sodium hydroxide or a nucleophilic catalyst exemplified by iodide can be added to the reaction system.

When the droplets have been converted to solid particles of adequate mechanical strength, the reaction mixture is filtered and the product collected and washed with solvents to remove the continuous phase, remaining starting material, by-products, and other contaminants.

Accordingly, in one embodiment there is provided a method for generating a cross-linked and beaded polymer matrix according to the invention, said method comprising the steps of

providing a poly(aminoalkylene) of Formula II and a cross-linking molecule of Formula III,
reacting under beading conditions the poly(aminoalkylene) and the cross-linking molecule, and
obtaining a cross-linked and beaded polymer matrix according to the invention.

The compounds of Formula II are preferably mixed with compounds of Formula III, optionally in the presence of a solvent. A surface active agent is present either in the mixed monomer phase or in the continuous phase. This mixture is subsequently added with stirring or ultrasonification to a liquid not miscible with the reactive mixture. The addition preferably involves a specific ratio of the reactants and a reaction temperature which ensures that the bead formation and cross-linking is fast. Optionally, nucleophilic or basic catalysts can also be present.

The stoichiometry of the reactants as defined by the molar ratio of nitrogen of Formula II to X of Formula III, (mol N/mol X), is preferably in the range of 500 to 0.1, such as 400 to 0.2, for example 300 to 0.3, such as 200 to 0.4, for example 100 to 0.5, such as 80 to 0.6, for example 70 to 0.7, such as 60 to 0.8, for example 50 to 0.9.

The cross-linking and beading process can be run neat or in the presence of a solvent, such as in water, methanol, ethanol, ethylene glycol, N,N-dimethylformamide, N,N-dimethylacetamide, N-methylpyrrolidone, or acetonitrile, or mixtures thereof.

The concentration of the reaction solution can be from 5 to 90%, such as from 10 to 80%, for example from 20 to 60%.

The stirring frequency is preferably from 1 to 2000 rpm, such as a stirring frequency of from 50 to 1000 rpm, such as from 100 to 800 rpm, for example from 100 to 600 rpm, such as from 100 to 500 rpm.

The non-miscible liquid is preferably a petroleum fraction, an aliphatic oil, a natural fat or triglyceride, an aromatic solvent such as toluene or xylene, a halogenated solvent such as methylene chloride, chloroform, carbon tetrachloride, dichloroethane, trichloroethylene, chlorobenzene, a fluorinated solvent, or mixtures thereof.

The ratio of the reactive phase and the non-miscible liquid is 10:1 to 1:10, such as from 5:1 to 1:5, for example from 2:1 to 1:2, or from 2:1 to 1:100, or from 4:5 to 1:75 or from 1:2 to 1:30.

The optional nucleophilic catalyst can be a salt such as sodium bromide, sodium iodide, potassium iodide, or tetrabutylammonium bromide.

The optional basic catalyst can be an alkaline compound such as sodium hydrogen carbonate, potassium carbonate, potassium hydroxide, or tetrabutylammonium hydroxide.

The optional surface active agent is preferably selected from the group consisting of:

negatively charged surface active agents such as, e.g., sodium laurate, sodium lauryl sulfate, sodium laurylsulfonate, sodium decylbenzenesulfonate;
neutral surface active agents such as, e.g., ethoxylated aliphatic alcohols, ethoxylated alkylphenols, alkylphenols, carbohydrate derived esters, e.g., sorbitan laurate, amphiphilic polymers such as copolymers of polyethylene glycol methacrylate and lauryl acrylate or trialkylsilylalkyl methacrylate or copolymers of ethylene oxide and propylene oxide, or homopolymers such as polyvinyl acetate or completely or partially hydrolysed polyvinyl acetate; and
positively charged surface active agents such as, e.g., hexadecyltrimethylammonium bromide, tetraheptyltrimethylammonium chloride, or tetrabutylammonium bromide.

The reaction temperature can be anything from −20° C. to 150° C., such as from 20° C. to 100° C., for example from 40° C. to 80° C.

In another embodiment of the invention there is provided a radical polymerisation method for the generation of beaded and cross-linked polymer matrices according to the invention. For use in this method, the polyamine, or derivatives thereof, comprises a chemical group able to react by radical polymerisation. The radically active starting material is subjected to bead forming conditions essentially as above. Thus, the material is dissolved in a solvent such as water, ethylene glycol, diethylene glycol, or dimethylformamide or mixtures thereof. A surface active agent and/or a radical initiator or a radical initiating system is preferably added to the reaction mixture or to the continuous system. This mixture is then added to a reactor containing a medium in which the reaction mixture is insoluble or essentially insoluble. The reactor is equipped with a stirring devise to efficiently form droplets of the reactive phase dispersed in the continuous phase. The temperature is adjusted to reach a reasonable reaction speed and reaction time. When the droplets have been converted to cross-linked particles of a desirable mechanical strength, the product is collected by filtration and washed with solvents to remove the continuous phase, remaining starting material, by-products, and other contaminants.

The radical polymerisable polyamine reactant can be prepared e.g. by acrylation, methacrylation, ethacrylation, maleamidation, or allylation of the polyamine or derivatives thereof. Suitable reagents for the making of radical polymerisable polyamines include e.g. acryloyl chloride, methacryloyl chloride, methacrylic acid anhydride, ethacryloyl chloride, maleic anhydride, and allyl chloride. The radical polymerisable polyamine is prepared by mixing the reactants, optionally in the presence of a solvent such as methylene chloride, or toluene, further optionally in the presence of a catalyst, such as a basic compound, such as an amine, for example triethylamine. When the reaction is completed, the optionally added solvent and/or catalyst is removed and the product can be used for radical polymerisation as described.

Accordingly, in this aspect of the invention there is provided a method for generating a cross-linked and beaded polymer matrix comprising the steps of:

providing a compound of Formula IV and a radical initiator,
reacting a reaction mixture as provided under a) under radical polymerisation conditions and beading conditions, and
obtaining a cross-linked and beaded polymer matrix according to invention.

The method can comprise the further step of providing a surface active agent, and/or a solvent, and/or a non-miscible phase to the reaction mixture, and reacting the reaction mixture under stirring or ultrasonification conditions and at a temperature allowing bead formation and cross-linking. Optionally, the surface active agent is added to the non-miscible phase.

A radical polymerization initiator can preferably be used to initiate the radical polymerization method. Examples of initiators include a peroxide, for example ammonium peroxodisufate, or tetrabutylammonium peroxodisulfate, a hydroperoxide such as t-butylhydroperoxide, an azo compound such as azoisobutyronitrile, a mixed initiator system such as a mixture of ammonium peroxodisulphate and sodium disulfite, or ammonium peroxodisulfate and N,N,N′,N′-tetramethyldiaminoethane, or ammonium peroxodisulfate, N,N,N′,N′-tetramethyldiaminoethane, and sodium disulfite.

The reaction temperature, the concentration of the reaction solution, the stirring frequency, the solvent, the non-miscible liquid, the surface active agent, and the ratio of the reactive phase and the non-miscible liquid is as described herein above.

When the polymer matrices are made by radical polymerization methods, there is further provided in accordance with the present invention a polymer matrix comprising a plurality of substituted amino groups, wherein the polymer matrix is obtained by a radical polymerization method as disclosed herein in combination with the further step of converting—after the polymerisation and beading steps—at least some of the amino groups to functional groups NR⁶R⁷, of Formula V:

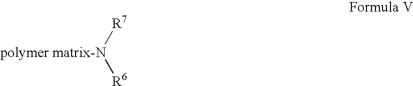

wherein R⁶ and R⁷ independently are H or an organic group formed by reaction of the amino groups of the polymer matrix according to the invention with an alkylating or acylating agent.

The alkylating agent is preferably an alkyl halide or a substituted alkyl halide, an alkyl sulphonate or a substituted alkyl sulphonate, an epoxide or a Michael electrophile.

Examples of alkylation agents in the form of optionally substituted alkyl halides include methyl iodide, ethyl iodide, propyl bromide, butyl bromide, chloroacetic acid, benzyl chloride, benzyl bromide, methylbenzyl bromide, methoxybenzyl bromide, or nitrobenzyl bromide.

Examples of alkylation agents in the form of alkyl sulphonates or a substituted alkyl sulphonates include methyl methanesulphonate, methyl trifluoromethanesulphonate, or methyl p-toluenesulphonate.

Examples of alkylation agents in the form of epoxides include ethylene oxide, propylene oxide, or a glycidol derivative thereof.

Examples of Michael electrophiles include methyl acrylate and ethyl acrylate.

The acylating agent is preferably

(a) an optionally activated carboxylic acid, such as formic acid, acetic acid, propionic acid, benzoic acid, mercaptoacetic acid, 3-mercaptopropanoic acid, thiolactic acid, protected aminoacids, such as N-(fluorenyloxymethylcarbonyl)glycine or N-(benzyloxycarbonyl)alanine, or N-(t-butoxycarbonyl)phenylalanine, or derivatives thereof, optionally activated by condensing agents such as dicyclohexylcarbodiimide,
(b) an activated carboxylic acid such as acetic anhydride, acetyl chloride, ethyl acetate, benzoyl chloride,
(c) a carbonic acid derivative such as methyl chloroformate, t-butyl chloroformate, benzyl chloroformate, or diphenyl carbonate, or
(d) a heteroallene such as ethyl isocyanate, phenyl isocyanate, ethyl isothiocyanate, or phenyl isothiocyanate.

The polymer matrix according to the invention preferably has a loading of functional groups in the range of from about 0.5 to about 33 mmol/g, such as from 1 to 20 mmol/g, for example from 2 to 15 mmol/g, such as from 2 to 10 mmol/g, for example from 2 to 8 mmol/g, such as from 2 to 6 mmol/g, for example from 2 to 4 mmol/g, such as from 4 to 15 mmol/g, for example from 6 to 15 mmol/g, such as from 8 to 15 mmol/g, for example from 10 to 15 mmol/g, such as from 12 to 15 mmol/g, for example from 2 to 6 mmol/g, such as from 6 to 10 mmol/g, for example from 10 to 14 mmol/g, such as from 14 to 18 mmol/g.

The polymer matrix according to the invention preferably has a swelling in an aqueous liquid, including water, in the range of 1 mL/g to 30 mL/g, such as from 1 mL/g to 20 mL/g, for example from 2 mL/g to 15 mL/g, such as from 3 mL/g to 10 mL/g, for example from 2 mL/g to 12 mL/g, such as from 2 mL/g to 10 mL/g, for example from 2 mL/g to 8 mL/g, such as from 2 mL/g to 6 mL/g, for example from 2 mL/g to 4 mL/g, such as from 4 mL/g to 20 mL/g, for example from 6 mL/g to 20 mL/g, such as from 8 mL/g to 20 mL/g, for example from 10 mL/g to 20 mL/g, such as from 12 mL/g to 20 mL/g, for example from 14 mL/g to 20 mL/g, such as from 16 mL/g to 20 mL/g, for example from 18 mL/g to 20 mL/g, for example from 2 mL/g to 6 mL/g, such as from 6 mL/g to 10 mL/g, for example from 10 mL/g to 14 mL/g, such as from 14 mL/g to 18 mL/g.

The beaded or granulated polymer matrix, or a composition comprising a plurality of beaded, cross-linked polymer matrices according to the invention preferably has an average particle diameter is in the range of 0.01 μm to 1500 μm, such as an average particle diameter is in the range of 10 to 1000 μm, for example an average particle diameter is in the range of 100 to 500 μm, such as about 200 μm, for example about 300 μm, such as about 400 μm.

The invention is also directed to the use of a beaded or granulated cross-linked polymer matrix comprising a plurality of functional groups selected from optionally substituted primary amines and secondary amines, preferably optionally substituted primary amines, for scavenging undesirable chemical compounds from a composition comprising a mixture of chemical entities. The undesirable chemical compounds are capable of reacting with the functional amine groups.

in one embodiment, the invention relates to the use of a granulated or beaded cross-linked polymer matrix comprising a plurality of functional groups selected from the group consisting of optionally substituted primary amines and secondary amines, preferably optionally substituted primary amines, for scavenging undesirable chemical compounds, preferably carbonyl and/or sulfonyl compounds, from a composition comprising a mixture of chemical entities, as support for immobilised reagents such as oxidizing agents, or alkylating agents, or complexing agents such as phosphines.

There is also provided the use of a polymer matrix according to the invention as described herein above for scavenging undesirable chemical compounds from a composition comprising a mixture of chemical entities.

The undesirable chemical compounds can e.g. be generated in organometallic reactions, but the use is not limited to such reactions.

The undesirable chemical compounds to be scavenged preferably comprise carbonyl and/or sulfonyl groups. Examples of such compounds include, but are not limited to, organic acids, acid chlorides, sulfonyl chlorides, ketones, aldehydes, and derivatives thereof.

The invention is also directed to the use of a beaded or granulated cross-linked polymer matrix comprising a plurality of functional groups selected from optionally substituted primary amines and secondary amines, preferably optionally substituted primary amines, for scavenging carbonyl compounds, such as e.g. acid chlorides, from a mixture containing such carbonyl compounds. The undesirable chemical compounds are capable of reacting with the functional amine groups.

There is also provided the use of a polymer matrix according to the invention as described herein above as a support for the synthesis of an organic molecule, or the use of a plurality of such matrices as a support for the generation of a combinatorial chemistry library comprising a plurality of different chemical entities.

In another embodiment there is provided the use of a polymer matrix according to the invention as described herein above as a support for the synthesis of a drug molecule, a peptide, a protein, DNA, or RNA.

In yet another embodiment there is provided the use of a polymer matrix according to the invention as described herein above as a support for solid phase enzyme reactions.

The matrices according to the invention can also be used for protein immobilisation, chromatographic separation and/or affinity purification of desirable target compounds having an affinity for the functional groups on the matrices according to the invention.

The invention is further described in the below examples which should not be construed as a limitation of the invention to the specific embodiments disclosed therein.

### EXAMPLES

### Example 1

The beaded polymer resin was prepared by an inverse suspension polymerization method. To a flask containing 10 g of water, 30 g polyvinylamine having a molecular weight of ˜50000 g/mol and 7.5 g diglycidyl ether poly(ethylene glycol) (Mₙ=400 g/mol) were added. After a homogenous solution had formed upon stirring the mixture, 0.75 g of a Castor oil ethoxylate was added to the solution. The reaction mixture was subjected to N₂ for 15 minutes. To a three-necked baffled flask, equipped with a mechanical stirrer, 600 mL of paraffin oil was added and heated to 70° C. The reaction mixture was added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove residuals and oil. The degree of amine functionality (amine capacity, loading) was analyzed to 3.9 mol/kg. The swelling performance in water was determined to 12 mL/g.

### Example 2

To a flask containing 20 g of water, 15 g polyvinylamine having a molecular weight of ˜50000 g/mol and 15 g diglycidyl ether poly(ethylene glycol) (Mₙ=400 g/mol) were added. Upon stirring the mixture, 0.60 g of a Castor oil ethoxylate was added to the solution. The reaction mixture was subjected to N₂ for 15 minutes. To a three-necked baffled flask, equipped with a mechanical stirrer, 600 mL of paraffin oil was added and heated to 70° C. The reaction mixture was added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove residuals and oil. The degree of amine functionality (amine capacity, loading) was analyzed to 1.7 mol/kg. The swelling performance in water was determined to 9 mL/g.

### Example 3

To a flask containing 13 g of water, 37.5 g polyvinylamine having a molecular weight of ˜50000 g/mol and 12.5 g diglycidyl ether poly(ethylene glycol) (Mₙ=400 g/mol) were added. The reaction mixture was subjected to N₂ for 15 minutes wherein a homogenous solution was formed upon stirring. To a three-necked baffled flask, equipped with a mechanical stirrer, 600 mL of paraffin oil was added and heated to 70° C. Following this, 0.5 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove residuals and oil. The degree of amine functionality (amine capacity, loading) was analyzed to 2.1 mol/kg. The swelling performance in water was determined to 9 mL/g.

### Example 4

To a flask containing 25 g of water, 234 g polyvinylamine (composed of a solid content of 24% dissolved in water) having a molecular weight of ˜50000 g/mol and 14.3 g diglycidyl ether poly(ethylene glycol) (Mₙ=400 g/mol) were added. The reaction mixture was subjected to N₂ for 20 minutes wherein a homogenous solution was formed upon stirring. To a three-necked baffled flask, equipped with a mechanical stirrer, 500 mL of paraffin oil was added and heated to 70° C. Following this, 1.1 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The degree of amine functionality (amine capacity, loading) was analyzed to 8.5 mol/kg. The compressed swelling performance in water was determined to 13 mL/g.

### Example 5

To a flask containing 3 g of water, 237 g polyvinylamine (composed of a solid content of 24% dissolved in water) having a molecular weight of ˜50000 g/mol and 6.4 g diglycidyl ether poly(ethylene glycol) (Mₙ=400 g/mol) were added. The reaction mixture was subjected to N₂ for 20 minutes wherein a homogenous solution was formed upon stirring. To a three-necked baffled flask, equipped with a mechanical stirrer, 450 mL of paraffin oil was added and heated to 70° C. Following this, 1.3 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The degree of amine functionality (amine capacity, loading) was analyzed to 9.9 mol/kg. The compressed swelling performance in water was determined to 40 mL/g.

### Example 6

To a flask containing 33 g of water, 198 g polyvinylamine (composed of a solid content of 24% dissolved in water) having a molecular weight of ˜50000 g/mol and 16.0 g diglycidyl ether poly(ethylene glycol) (Mₙ=400 g/mol) were added. The reaction mixture was subjected to N₂ for 20 minutes wherein a homogenous solution was formed upon stirring. To a three-necked baffled flask, equipped with a mechanical stirrer, 450 mL of paraffin oil was added and heated to 70° C. Following this, 1.3 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The degree of amine functionality (amine capacity, loading) was analyzed to 6.9 mol/kg. The compressed swelling performance in water was determined to 10 mL/g.

### Example 7

To a flask containing 25 g of water, 234 g polyvinylamine (composed of a solid content of 24% dissolved in water) having a molecular weight of ˜50000 g/mol and 14.3 g diglycidyl ether poly(propylene glycol) (Mₙ=380 g/mol) were added. The reaction mixture was subjected to N₂ for 20 minutes wherein a homogenous dispersion was formed upon stirring. To a three-necked baffled flask, equipped with a mechanical stirrer, 500 mL of paraffin oil was added and heated to 70° C. Following this, 1.1 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The compressed swelling performance was determined to 6.0 mL/g in water, and 6.6 mL/g in ethanol.

### Example 8

To a flask containing 61 g of water, 211 g polyvinylamine (composed of a solid content of 24% dissolved in water) having a molecular weight of ˜50000 g/mol and 6.9 g triglycidyl ether poly(ethylene glycol) (Mₙ=100 g/mol) were added. The reaction mixture was subjected to N₂ for 20 minutes wherein a homogenous dispersion was formed upon stirring. To a three-necked baffled flask, equipped with a mechanical stirrer, 400 mL of paraffin oil was added and heated to 70° C. Following this, 0.9 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The compressed swelling performance was determined to 7.6 mL/g in water, and 7.6 mL/g in ethanol.

### Example 9

To a flask containing 61 g of water, 211 g polyvinylamine (composed of a solid content of 24% dissolved in water) having a molecular weight of ˜50000 g/mol and 5.7 g triglycidyl ether poly(ethylene glycol) (Mₙ=500 g/mol) were added. The reaction mixture was subjected to N₂ for 20 minutes wherein a homogenous dispersion was formed upon stirring. To a three-necked baffled flask, equipped with a mechanical stirrer, 400 mL of paraffin oil was added and heated to 70° C. Following this, 0.9 g of an oil-soluble polymeric surfactant was added and dissolved in the oil. The reaction mixture was then added to the oil forming beads. The chemical synthesis, i.e. network formation, was performed at 70° C. for 20 h. After the synthesis, the resulting beads were filtrated from the oil phase. The beads were then sequentially washed with dichloromethane, tetrahydrofurane, methanol and water to remove rest-products and oil. The compressed swelling performance was determined to 7.1 mL/g in water, and 7.4 mL/g in ethanol.

### Example 10

### Scavenging of Palladium

In order to test the ability to scavenge palladium ions form acidic polar solvents the polyvinylamine resins were tested under a range of conditions. A 0.5% (W/W) solution of Palladium(II) chloride in 1M HCl in a water ethanol mixture (1:1) was prepared.

2 mL of this solution were added to each of a number of small vials. The vials were added amounts varying from 0 to 10 mg of dry resin prepared according to Example 1, shaken for 60 minutes, and the colour intensity of the supernatant was noted. The results are summarized in the following scheme.

| Amount of resin added | Colour |
|---|---|
| 0 mg | Yellow brown |
| 1 mg | Weak yellow brown |
| 2 mg | Faint yellow |
| 5 mg | Colorless |
| 10 mg | Colorless |

1. A beaded polymer matrix, formed by cross-linking of optionally substituted poly(aminoalkylene), under inverse suspension or inverse emulsion polymerisation conditions, of Formula I 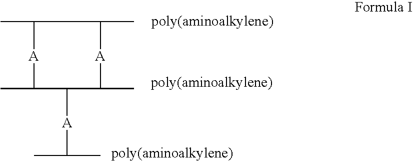
wherein A is a cross-linking unit of functionality ≧2,
with the proviso that when poly(aminoalkylene) is poly(allylamine) then at least 1% of all nitrogens are substituted, and with the further proviso that when poly(aminoalkylene) is poly(vinylamine) then A is not (a) a polymethylene of the formula (CH₂)ᵣ wherein r is an integer from 2 to 10, or (b) an optionally substituted xylylene, or (c) a diimine linked by a polymethylene of the formula (CH₂)ₛ wherein s is an integer from 2 to 5, or (d) an optionally substituted xylylene, or (e) CH₂CHOHCH₂ or CH₂CHCH₂OH.

2. The beaded cross-linked poly(aminoalkylene) matrix according to claim 1, wherein said poly(aminoalkylene) is of Formula II 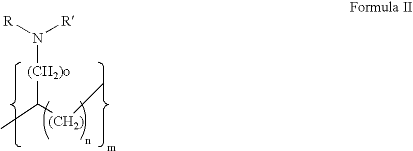
wherein
R and R′ independently are selected from the group consisting of hydrogen, optionally substituted alkyl, optionally substituted aryl groups, and optionally substituted acyl groups;
n is a number from 0 to 10;
m is a number from 3 to 15000;
herein o is number 0 or 1

3. The beaded cross-linked poly(aminoalkylene) according to claim 1 wherein poly(aminoalkylene) is optionally substituted poly(aminomethylene), optionally substituted polyvinylamine, or substituted poly(allylamine).

4. (canceled)

5. (canceled)

6. The beaded cross-linked poly(aminoalkylene) matrix according to claim 1 wherein the cross-linking unit A is obtained by reacting a poly(aminoalkylene) with a cross-linking molecule of Formula III
AX_{q} Formula III
wherein A is saturated or unsaturated aliphatic and/or aromatic or composed of both saturated and/or unsaturated aliphatic and aromatic fragments, and optionally containing heteroatoms such as silicon, nitrogen, phosphorous, oxygen, or sulphur;
wherein X is a reactive group;
wherein q, is the number of reactive groups, such as 2, 3, 4, 5, or 6;
with the proviso that when poly(aminoalkylene) is poly(vinylamine) then AX_{q} is not
(a) a dibrominated or diiodated polymethylene expressed by general Formula (2) 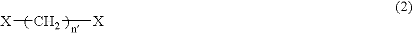
where X denotes Br or I, and n′ denotes an integer of 2 to 10), or
(b) a p-dihalogenated xylylene expressed by general Formula (3) 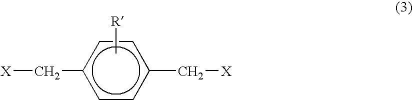
where X denotes Cl, Br, or I; and R′ denotes H, a methyl group, an ethyl group, or a halogen atom, or
(c) a nuclear-substituted derivative thereof as the polyfunctional cross-linking agent that can bond with alkyl-substituted primary amino groups, or
(d) a polymethylene dialdehyde expressed by general Formula (4) 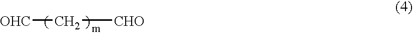
where m denotes an integer of 2-5, or
(e) a dialdehyde having an intramolecular benzene nucleus expressed by general Formula (5) 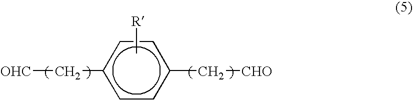
where I denotes 0 or an integer of 1-20, and R′ denotes H, a methyl group, an ethyl group, or a halogen atom, or
(f) epichlorohydrin.

7. The beaded cross-linked poly(aminoalkylene) matrix according to claim 6 wherein A is an aliphatic or alkylaryl group having 2 to 200 carbon atoms and optionally having 1 to 100 hetero atoms such as nitrogen, oxygen, or sulphur.

8. The beaded cross-linked poly(aminoalkylene) matrix according to claim 6 wherein the cross-linking molecule AX_{q} is
a) ethylene dibromide, propylene dibromide, butylene dibromide, xylylene dibromide, ethylene glycol ditosylate, diethylene glycol dichloride, triethyleneglycol dichloride, polyethylene glycol dichloride, epichlorohydrine, ethylene glycol diglycidyl ether, diethylene glycol diglycidyl ether, triethylene glycol diglycidyl ether, polydisperse polyethylene glycol diglycidyl ether such as (ethylene oxide)₁₀, diglycidyl ether, (ethylene oxide)₁₅, diglycidyl ether (ethylene oxide)₂₀, diglycidyl ether, ethoxylated trimethylolpropane triglycidyl ether, ethoxylated dipentaerythritol hexaglycidyl ether, with the proviso that when Ax_{q} is ethylene dibromide, propylene dibromide, butylene dibromide, xylylene dibromide then poly(aminoalkylene) is not an optionally substituted polyvinylamine;
b) ethylene glycol diacrylate, diethyleneglycol diacrylate, polyethylene glycol diacylate, polyethyleneglycol dimethacrylate, ethoxylated trimethylolpropane triacrylate, ethoxylated dipentaerythritol hexaacrylate, or Jeffamine diacrylate;
c) 1,6-hexane diisocyanate, isophorone diisocyanate, toluene diisocyanate, or 1,4-phenylene diisocyanate; or
d) formaldehyde, glyoxal, succinaldehyde, glutaraldehyde, 1,4-diformylbenzene, 1,4-diacetylbenzene, polyethylene glycol di(formylmethyl)ether with the proviso that the cross-linking step is followed by reduction of the imine to the amine.

9. A beaded cross-linked poly(aminoalkylene) matrix obtained by radical polymerization of a molecule of Formula IV having a radical reactive group R⁴R′″C═CR″—CY 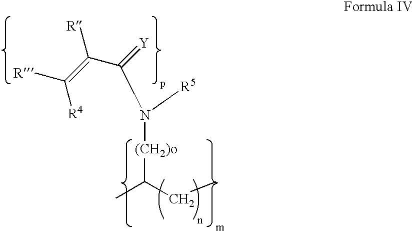
wherein
n is a number from 0 to 10;
m is a number from 3 to 15000;
o is number 0 or 1;
p is a number >0 and <m;
Y is a heteroatom or a pair of hydrogens; and
R″, R′″, R⁴, and R⁵ are independently selected from the group consisting of hydrogen, optionally substituted saturated or unsaturated alkyl, optionally substituted saturated or unsaturated acyl, and optionally substituted aryl groups.

10. The beaded cross-linked poly(aminoalkylene) matrix according to claim 9, obtained by radical polymerization, wherein poly(aminoalkylene) comprises poly(aminomethylene), polyvinylamine, or poly(allylamine).

11. The beaded cross-linked poly(aminoalkylene) matrix obtained by radical polymerization, according to claim 9, wherein the reactive group R⁴R′″C═CR″—CY is acryloyl, methacryloyl, ethacryloyl, or allyl.

12. A method generating a cross-linked and beaded polymer matrix according to claim 1 comprising the steps of:
a) providing a poly(aminoalkylene) of Formula II and a cross-linking molecule of Formula III,
b) reacting under beading conditions the poly(aminoalkylene) and the cross-linking molecule,
c) obtaining a cross-linked and beaded polymer matrix according to claim 1.

13. A method for generating a cross-linked and beaded polymer matrix according to claim 1 comprising the steps of:
a) providing a compound of Formula IV and a radical initiator,
b) reacting a reaction mixture as provided under a) under radical polymerisation conditions and beading conditions,
c) obtaining a cross-linked and beaded polymer matrix according to claim.

14. The method of claim 13, comprising the further step of providing a surface active agent, and/or a solvent, and/or a non-miscible phase to the reaction mixture, and reacting the reaction mixture under stirring or ultrasonification conditions at a temperature allowing bead formation and cross-linking.

15. A polymer matrix comprising a plurality of substituted amino groups wherein the polymer matrix is obtained by the method of claim 12 comprising the further step of converting at least some of the amino groups after the polymerisation and beading steps to functional groups NR⁶R⁷, of Formula V: 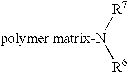
wherein R⁶ and R⁷ independently are selected from hydrogen and an organic group formed by reaction of the amino groups of the polymer matrix according to claim 1 with an alkylating or acylating agent.

16. Use of a granulated or beaded cross-linked polymer matrix comprising a plurality of functional groups selected from the group consisting of optionally substituted primary amines and secondary amines for scavenging undesirable chemical compounds from a composition comprising a mixture of chemical entities, as support for immobilised.

17. Use of the polymer matrix according to claim 1 for scavenging undesirable chemical compounds from a composition comprising a mixture of chemical entities.

18. Use of the polymer matrix according to claim 1 as support for the synthesis of an organic molecule.

19. Use of a plurality of polymer matrices according to claim 1 as supports when generating a combinatorial chemistry library.

20. Use of a plurality of polymer matrices according to claim 1 as supports when generating a library of chemical entities.

21. Use of the polymer matrix according to claim 1 as a support for the synthesis of a drug molecule, a peptide, a protein, DNA, or RNA.

22. Use of the polymer matrix according to claim 1 as support for solid phase enzyme reactions.

23. Use of the polymer matrix according to claim 1 for protein immobilisation of biomolecules.

24. Use of the polymer matrix according to claim 1 for chromatographic separation or purification of desirable target compounds including affinity purification.
